(19) United States
(12) Patent Application Publication
(10) Pub. No.: US 2018/0133094 A1
(43) Pub. Date: May 17, 2018

(54) MEDICAL TRANSPORTER

(71) Applicant: Global Medical Mentors Inc., Jacksonville, FL (US)

(72) Inventor: Zev HELFER, Jacksonville, FL (US)

(21) Appl. No.: 15/814,513

(22) Filed: Nov. 16, 2017

Related U.S. Application Data
(60) Provisional application No. 62/422,653, filed on Nov. 16, 2016.

(51) Int. Cl. A61H 3/04

(52) U.S. Cl. CPC A61H 3/04; A61M 5/1415

(57) ABSTRACT

A medical transporter and methods of use thereof by medical patients and other persons needing assistance for walking and/or moving about. The medical transporter includes a frame that surrounds a person and allows for protection of a person and for stability of the person during transport of the patient. The medical transporter includes a stand for mounting an IV bag. In preferred embodiments, a removable seat, foldable tray and basket are provided.

### CROSS REFERENCE TO RELATED APPLICATIONS

This application claims priority to U.S. Provisional Patent Application No. 62/422,653 entitled Medical Transporter filed Nov. 16, 2016. The content of this application is incorporated by referenced herein in its entirety.

### FIELD OF THE INVENTION

A medical transporter and methods of use thereof by medical patients and other persons needing assistance for walking and/or moving about. The medical transporter includes a frame that surrounds a person and allows for protection of a person and for stability of the person during transport of the patient.

### BACKGROUND OF THE INVENTION

Walking is one of the most crucial things that a person can do to prevent problems. Walking promotes the flow of oxygen throughout a person's body and maintains normal breathing function. Walking also strengthens muscle tone, improves gastrointestinal and urinary tract function, improves blood flow and speeds wound healing.

For people that have undergone surgery and/or are elderly, frail and/or have suffered from injuries, additional devices are required to help such persons or patients to walk. Medical walkers and walking aid devices are used to assist patients in a hospital or patients suffering from injuries to walk and or move about with support from the walker or walking aid device. Known systems include four-leg push-along walkers with wheels in the front of the walker (at least one wheel on each front leg), four-leg push-along walkers (at least one wheel on each leg), crutches and rollator devices.

However, these known systems suffer from several disadvantages. In particular, each of these known systems suffer from several disadvantages for helping a person with limited mobility and/or ability.

Each of the existing systems are designed for patients that have a minimum amount of strength and/or are able to support themselves and/or some of their weight by their arms. However, physically frail or weak patients are generally unable to adequately support themselves using their arms for extended periods; therefore, use of existing walkers and walking aid devices is difficult for the elderly, frail, or those who labor with neurological difficulties or congenital deformity.

Existing systems are also difficult to be used with a healthcare worker such as a nurse, as current four-leg push-along walkers do not allow for the healthcare worker to help support the patient and if the patient begins to fall there is no support for the patient. Known four-legged walkers typically do not surround a patient and provide no support or structure to the rear of a patient, and pose a further safety risk in that the walker can be “thrown out in front” of a patient if they happen to lose balance and begin to fall. This can result in injury to a healthcare worker if the patient ends up falling on the healthcare worker.

Moreover, most existing systems do not include a safety harness or seat which will catch the patient in the event of a fall.

In view of disadvantages in existing systems, healthcare workers often transport patients in a wheelchair. Use of such a wheelchair is often recommended or prescribed for a patient who is incapable of safely walking or moving about with an existing medical walker or a walking aid. However, consistent use of a wheelchair can lead to further physical decline caused by being sedentary and results in comorbid muscle atrophy.

Accordingly, it is desirable to provide a medical transporter or medical walker which solves the disadvantages of current four legged walkers.

It is also desirable to provide a medical transporter which is safe and easy to maneuver, and which allows for the use of the muscles in the arms and legs of patients who are able to walk, while being fully protected and eliminating the danger of the patients falling to the ground.

### SUMMARY OF THE INVENTION

Accordingly, it is an object of the invention to provide a medical transporter which is both safe and relatively easy to maneuver.

It is another object of the invention to provide a medical transporter that surrounds a patient that allows for increased safety when transporting patients, both for the patients and for healthcare workers.

It is another object of the invention to provide a medical transporter that helps reduce injury to healthcare workers, which can result during transport of patients.

It is another object of the invention to provide a medical transporter which is relatively inexpensive to manufacture, and relatively easy to construct.

It is another object of the invention to provide a medical transporter that acts as a hybrid between a wheelchair and a transporter or transporter aid device.

It is another object of the invention to provide a medical transporter that allows for patients to be comfortable while being transported, transported with dignity, and reduces the fear of patients of falling or fainting while being transported.

It is another object of the invention to provide a medical transporter that is safer, so as to reduce injuries and encourages patients to walk around more often, which results in patients becoming stronger and leaving hospitals sooner.

It is another object of the invention to provide a medical transporter that is designed to allow for mobilization of post-surgical and hospital patients to move under their own power, rather than being “walked” by two to three hospital employees.

It is another object of the invention to provide a medical transporter for children and/or adults.

These and other objects of the present invention are achieved by providing a medical transporter according to the claims.

These and other objects of the present invention are achieved by providing a medical transporter for transportation of a patient, comprising: a frame, the frame configured to entirely enclose a patient during use of the medical transporter, said frame comprising: a first frame member, the first frame member having an upper support member and a lower support member, the lower support member connected to a pair of wheels, a second frame member, the second frame member having an upper support member, and a lower support member, the lower support member connected to a pair of wheels, a hinge tethered on one end to the first frame member and on the other end to the second frame member, the hinge allowing the first frame member to pivot with respect to the second frame member, a lock ring tethered on one end to the first frame member, the lock ring having a flexible receiving portion configured to interface with the second frame member, such that the lock ring holds the first frame member to the second frame member during use of the medical transporter; and a removable stand, the removable stand being configured to support an IV drip bag, wherein the patient is able to transport the IV drip bag through transport of the medical transporter.

In certain embodiments, the medical transporter further comprises a removable seat, wherein the removable seat is secured to the first frame member and to the second frame member.

In certain embodiments, the upper support member of the first frame member and the upper support member of the second frame member have connection members, the connection members configured to support the removable seat and the weight of the patent.

In certain embodiments, the connection members are selected from the group consisting of hooks and rings.

In certain embodiments, the removable seat is made a pliable polyester material or plastic material that is deformable and is configured to adapt to support the torso of the patient.

In certain embodiments, the seat does not catch the torso of the patient. In certain embodiments, the seat is closer to the buttocks area of a patient.

In certain embodiments, the removable seat may be attached or detached depending upon the patient. In certain embodiments, the removable seat is totally collapsible between the legs of a patient while the patient is walking. In certain embodiments, the seat is only used in case of emergency to prevent falls or fainting.

In certain embodiments, the removable seat includes an optional disposable diaper. In certain embodiments, the disposable diaper is custom fitted to seats.

In certain embodiments, the seats come in various sizes for various sizes of patients. In certain embodiments, the materials used for the removable seat are flexible, breathable, may be autoclaved, washed, treated for microbes.

In certain embodiments, the wheels include wheels with two locking castors and two non-locking castors. In certain embodiments, the wheels secure entry and exit of device.

In certain embodiments, the wheels include wheels with four locking castors.

In certain embodiments, the wheels are self-braking without hand brakes. In certain embodiments, hand brakes are provided to assist with the braking of the wheels.

In certain embodiments, the wheels are all terrain wheels, such that the wheels are designed to be indoor and outdoor wheels and are designed to go over pavement, gravel, grass and/or dirt.

In certain embodiments, the frame is made of a metal, a metal alloy, steel or a material configured to support a patient up to 350 lbs. in certain embodiments, the frame is made of at least one vertical support member, such that the weight of a patient is supported, especially if the patient is heavy and/or weights up to 350 lbs or even more.

In certain embodiments, the upper frame member includes a top support frame member and a middle support frame member, the top support frame member and the middle support frame member connected by at least one vertical support members.

In certain embodiments, the top support member and the middle support member are hollow steel tubes.

In certain embodiments, the top support member and the middle support member have at least some curved portions.

In certain embodiments, the medical transporter further comprises elastic straps, the elastic straps configured to connect the removable seat to the first frame member and to the second frame member.

In certain embodiments, the lower support member of the first frame member and the lower support member of the second frame member each comprise at least one reinforced structural component, the at least one reinforced structural component configured to provide additional support for the weight of the patient.

In certain embodiments, the at least one reinforced structural component include at least one vertical structural support members.

In certain embodiments, at least one of the pair of wheels on the first frame member and the second frame member have locking members.

In certain embodiments, the locking member is controlled via an actuator element located on the first frame member or the second frame member.

In certain embodiments, the upper support member of the first frame member is curved and/or has a U-shape.

In certain embodiments, the upper support member of the second frame member is curved and/or has a U-shape.

In certain embodiments, the hinge includes a hinge pin. In certain embodiments, the hinge pin includes a loop at one end, the loop being fitted around the upper support member.

In certain embodiments, the hinge is welded into the first frame member and to the second frame member.

In certain embodiments, the removable stand is fitted to interface with the first or the second frame member. In certain embodiments, the removable stand in configured to slide within the upper support member or within a reinforcement member in the frame.

In certain embodiments, the removable stand is disappearing, such that it is configured to be adjusted into the frame.

In certain embodiments, the upper support member of the first frame member and the upper support member of the second frame member comprises a crossbar connected via the at least one reinforced structural component, wherein the at least one reinforced structural element is oriented vertically.

In certain embodiments, the frame is configured to rotate or pivot from an open state to a closed state.

In certain embodiments, the frame is adjustable with respect to its overall circumference and height which allows the patient as well as medical staff to safely and effectively use the medical transporter.

In certain embodiments, the medical transporter completely supports the weight of the patient, and prevent the patients from falling to the ground resulting in an accident or medical emergency.

In certain embodiments, the medical transporter further comprises a seat harness suspended from the patient support frame which acts to catch and support the patient in the event of a fall.

In certain embodiments, the medical transporter includes an adjustable frame.

In certain embodiments, the medical transporter is made from stainless steel. In certain embodiments, the medical transporter is made from a mixed polymer injection mold. In certain embodiments, the medical transporter may be screen printed and/or may have logos or personalization printed or attached onto the transporter.

In certain embodiments, the medical transporter comes in many sizes. Certain sizes may be pediatric, medium, large, or obese. In certain embodiments, the medical transporter may be custom ordered for specific size and height.

In certain embodiments, the device may have solid legs that are no adjustable.

In certain embodiments, the medical transporter includes at least one removable stand for mounting IV (intravenous) bags, dosing pumps, oxygen tanks and/or concentrators, medical accessories and the like.

In certain embodiments, the medical transporter includes a shelf for allowing for holding a mountable IV bag, so that the mountable IV bag can be replaced.

In certain embodiments, the shelf is fixed to the frame of the medical transporter. In certain embodiments, the shelf is removable from the frame of the medical transporter. In certain embodiments, shelf can be placed into a locked and unlocked position, such that in the locked position, the shelf is unable to pivot.

In certain embodiments, the medical transporter is configured to fit through all standard doors. In certain embodiments, the medical transporter is waterproof and/or may be used in showers.

In certain embodiments, the medical transporter allows for front or rear access into the device.

In certain embodiments, the medical transporter reduces falls by persons 100% or approximately 100%.

In certain embodiments, the distance between the upper support member of the first frame member and the upper support member of the second frame member is approximately equal to the distance between the lower support member of the first frame member and the lower support member of the second frame member.

In certain embodiments, the frame is configured to support various devices needed by the patient during transport of the patient.

Other objects of the invention are achieved by providing a medical transporter for transportation of a patient, comprising: a frame, the frame configured to surround a patient during use of the medical transporter, said frame comprising: a first frame member, the first frame member having an upper support member, a lower support member and a vertical standard, the lower support member connected to a pair of wheels and the upper support member connected to the lower support member via the vertical standard, a second frame member, the second frame member having an upper support member, a lower support member and a vertical standard, the lower support member connected to a pair of wheels, and the upper support member connected to the lower support member via the vertical standard, a hinge tethered on one end to the first frame member and on the other end to the second frame member, the hinge allowing the first frame member to pivot with respect to the second frame member, and a locking element for releasably locking said patient support frame in a closed position during use of the medical transporter.

In certain embodiments, the transporter includes a first locking strap attached to the vertical standard of the first frame member, and a second locking straps attached to the vertical standard of the second frame member, the first strap locking strap having a plurality of hook members located on an inner surface and a second locking strap having a plurality of complementary loop members located on an outer surface, whereby the first locking strap and the second locking straps are locked together by mating the hook members of the first locking strap with the loop members of the second locking strap.

In certain embodiments, the transporter includes a removable stand, the removable stand being configured to support an IV drip bag, wherein the patient is able to transport the IV drip bag through transport of the medical transporter.

Other objects of the invention are achieved by providing a method of transporting a patient in need thereof comprising: inserting a patient into the medical transporter of claim 1, wherein the patient is inserted into the transporter when the transporter is in an open position; closing the medical transporter around the patient; and transporting the patient from a first location to a second location.

Other objects of the invention are achieved by providing a method of using the medical transporter as described in the specification.

Other objects of the invention and its particular features and advantages will become more apparent from consideration of the following drawings and accompanying detailed description. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is a perspective view of an embodiment of the medical transporter in a closed position;

FIG. 2 is a perspective view of the medical transporter of FIG. 1 in an open position;

FIG. 3 is a perspective view of the medical transporter of FIG. 1 in a disassembled or separated configuration;

FIG. 4 is a perspective view of the medical transporter of FIG. 1 in a closed position detailing the removable stand;

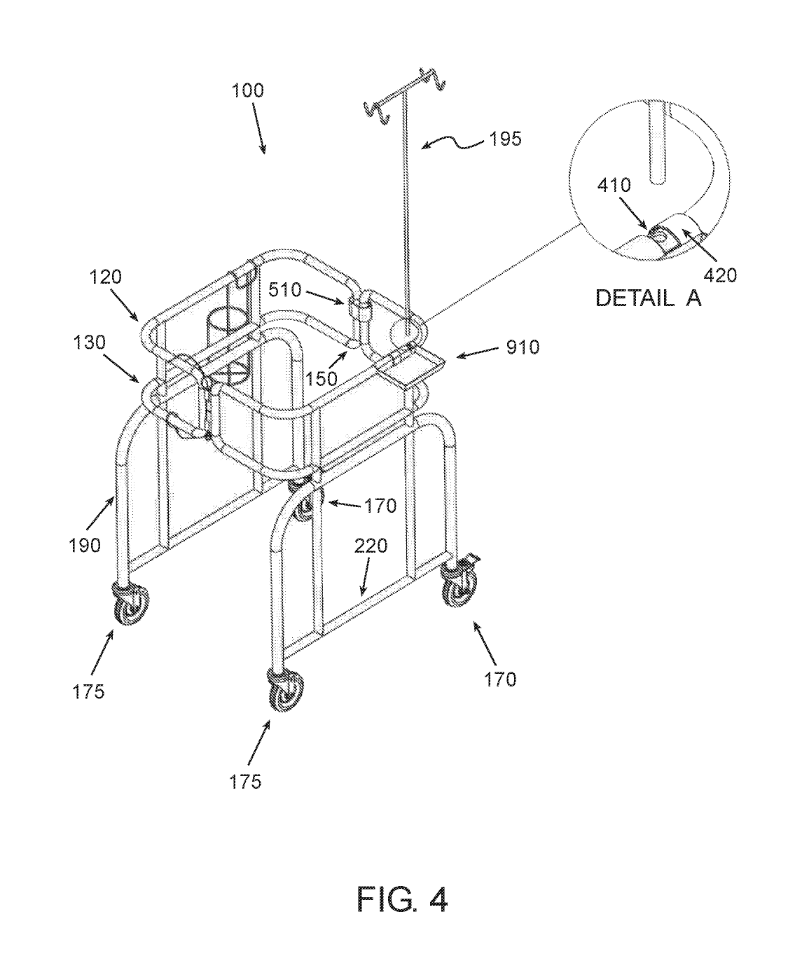

FIG. 5 is a perspective view of the medical transporter of FIG. 1 in a disassembled or separated configuration detailing a hinge;

FIG. 6 is a perspective view of the medical transporter of FIG. 1 in a disassembled or separated configuration detailing a hinge pin;

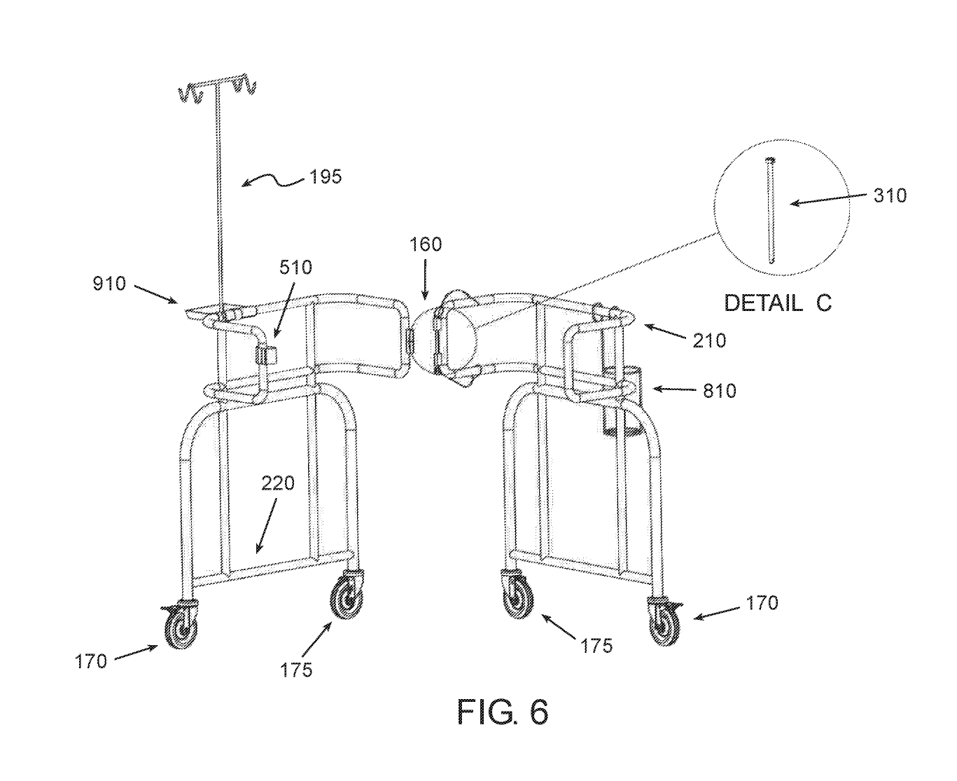

FIG. 7 is a perspective view of the medical transporter of FIG. 1 in a disassembled or separated configuration detailing a hinge and hinge pin connection;

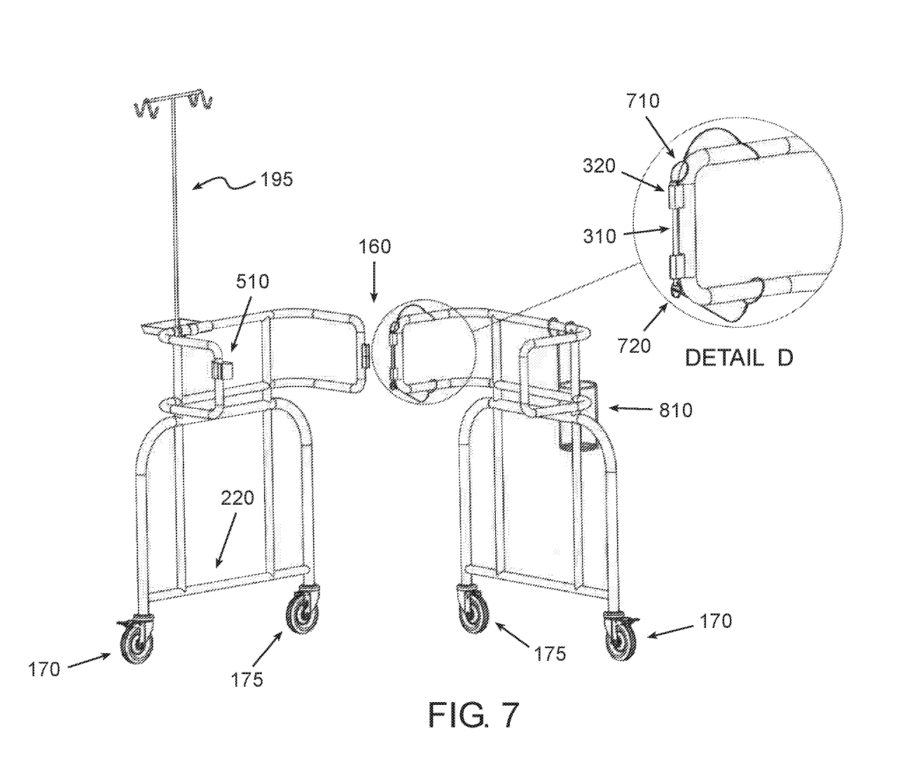

FIG. 8 is a perspective view of the medical transporter of FIG. 1 in a disassembled or separated configuration detailing a locking element;

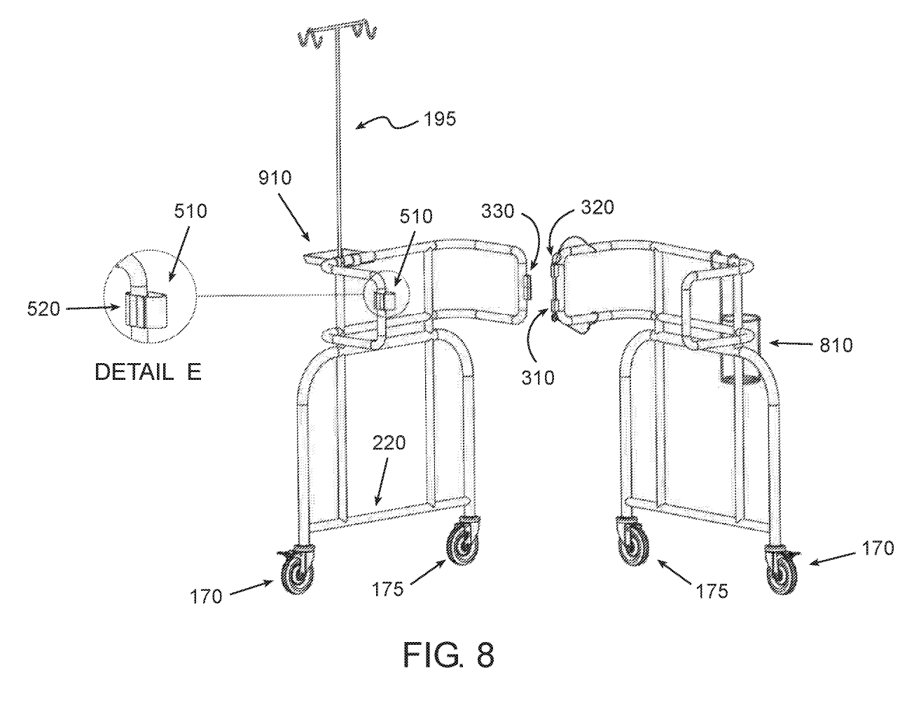

FIG. 9 is a perspective view of the medical transporter of FIG. 1 in a closed position and detailing the foldable tray;

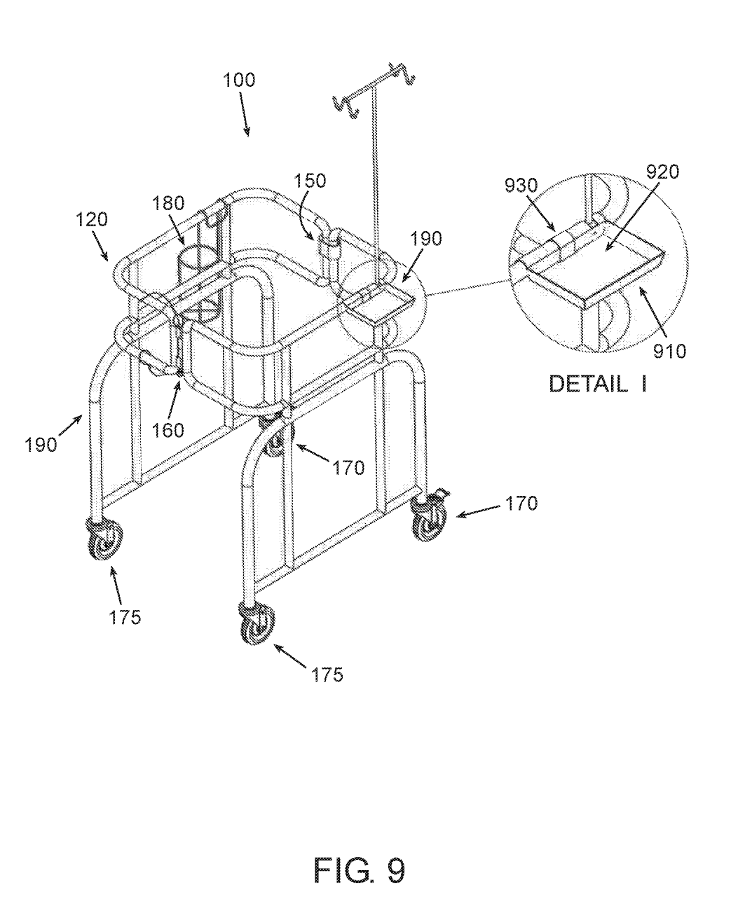

FIG. 10 is a perspective view of the medical transporter of FIG. 1 in a closed position and detailing the basket;

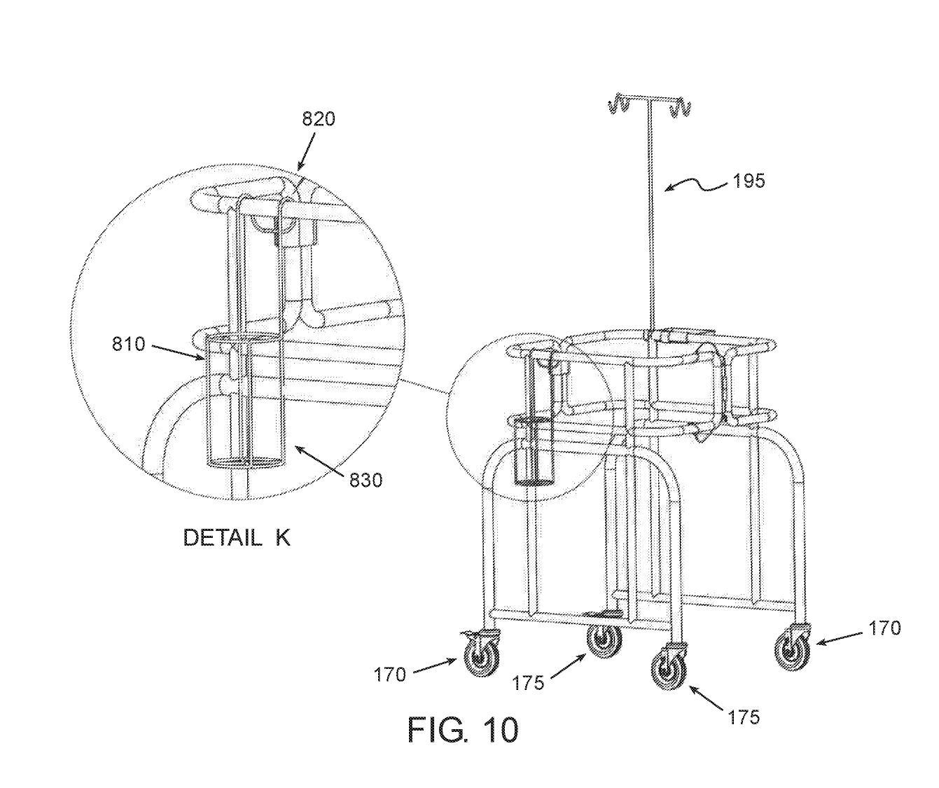

FIG. 11 is a perspective view of the medical transporter of FIG. 1 in an open configuration depicting a seated patient preparing the medical transporter for use.

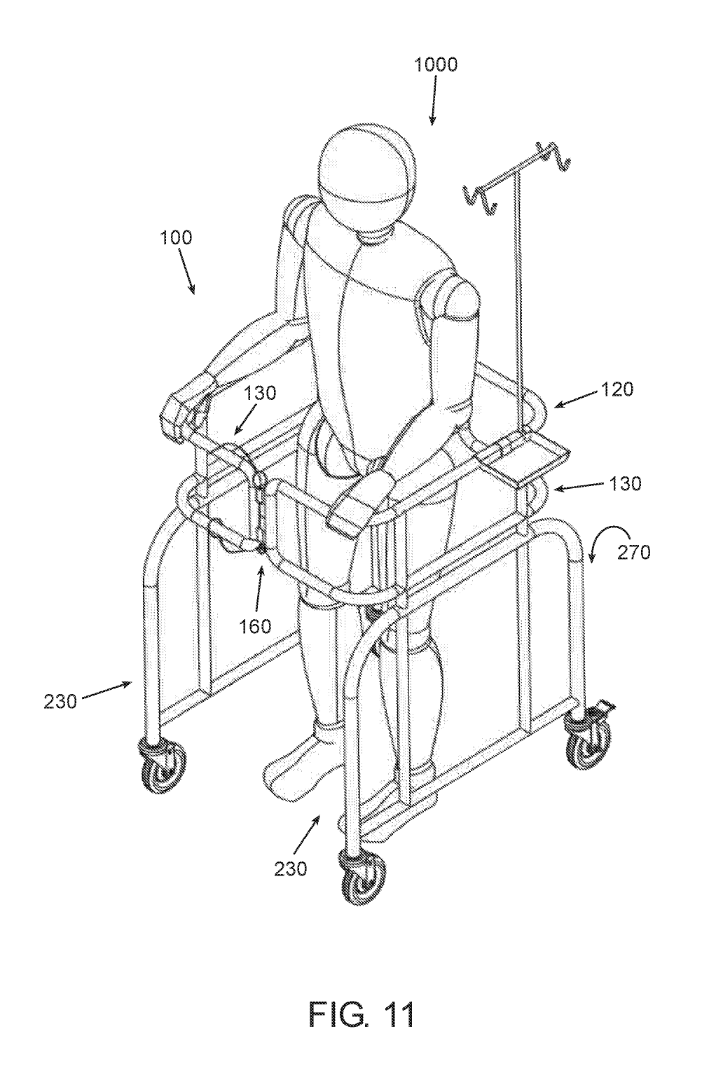

FIG. 12 is a perspective view of the medical transporter of FIG. 1 in a closed configuration depicting a standing patient using the medical transporter;

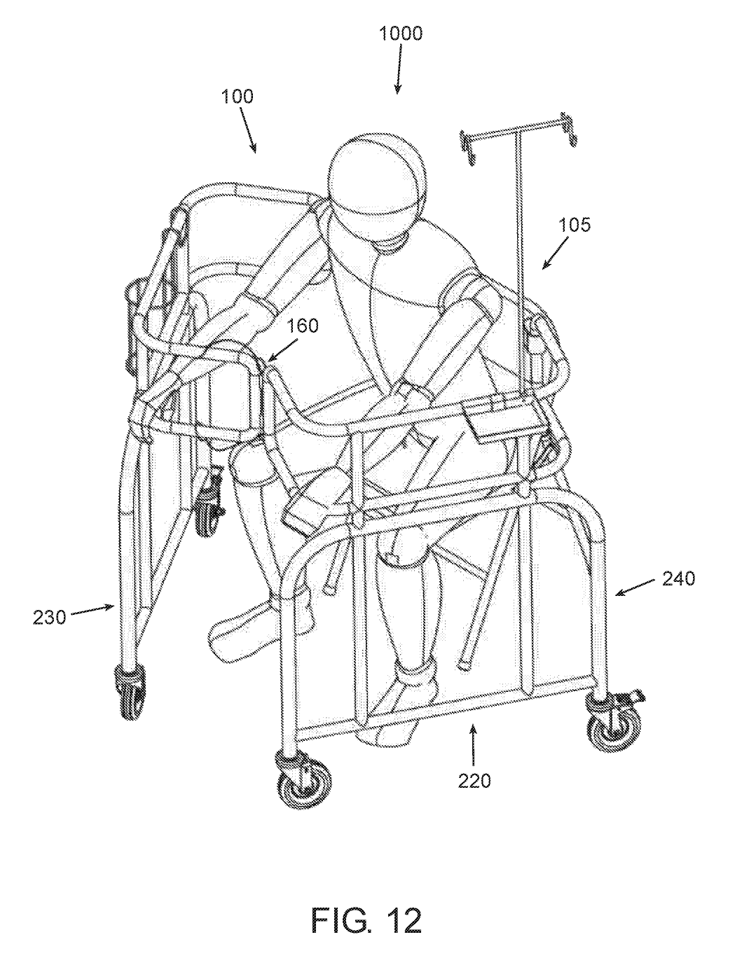

FIG. 13 is a perspective view of an embodiment of the medical transporter in a closed position;

FIG. 14 is a perspective view of the medical transporter of FIG. 13 in a closed configuration depicting a standing patient using the medical transporter;

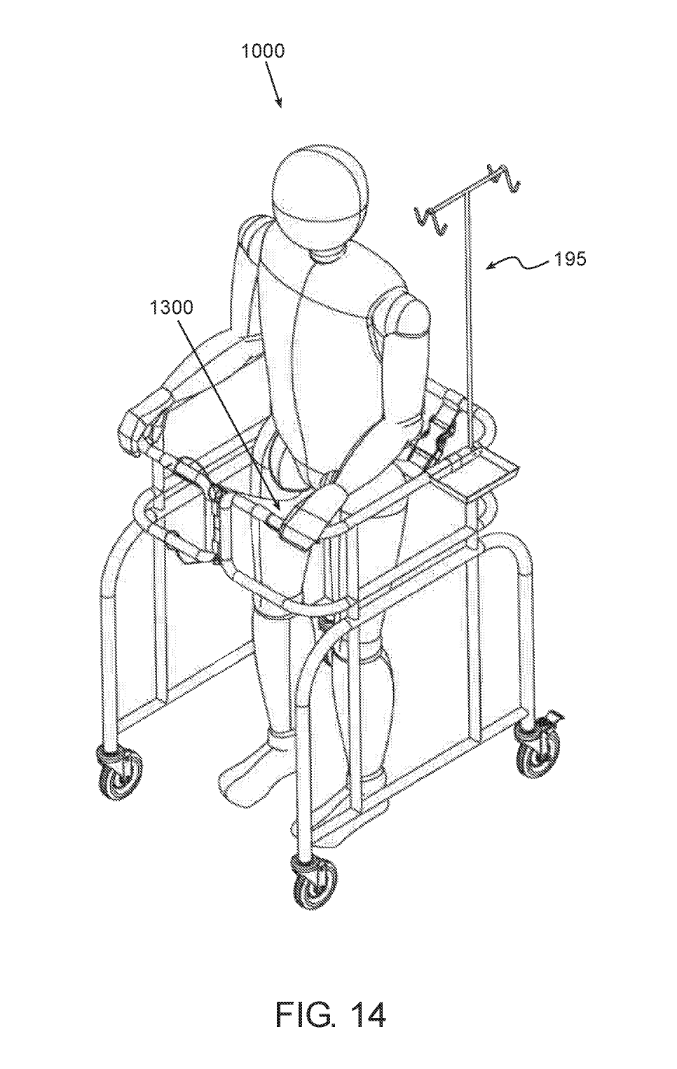

FIG. 15 is a perspective view of the medical transporter of FIG. 13 in a closed configuration detailing the seat; and

FIG. 16 is a perspective view of the medical transporter of FIG. 13 in a closed configuration detailing the seat and basket.

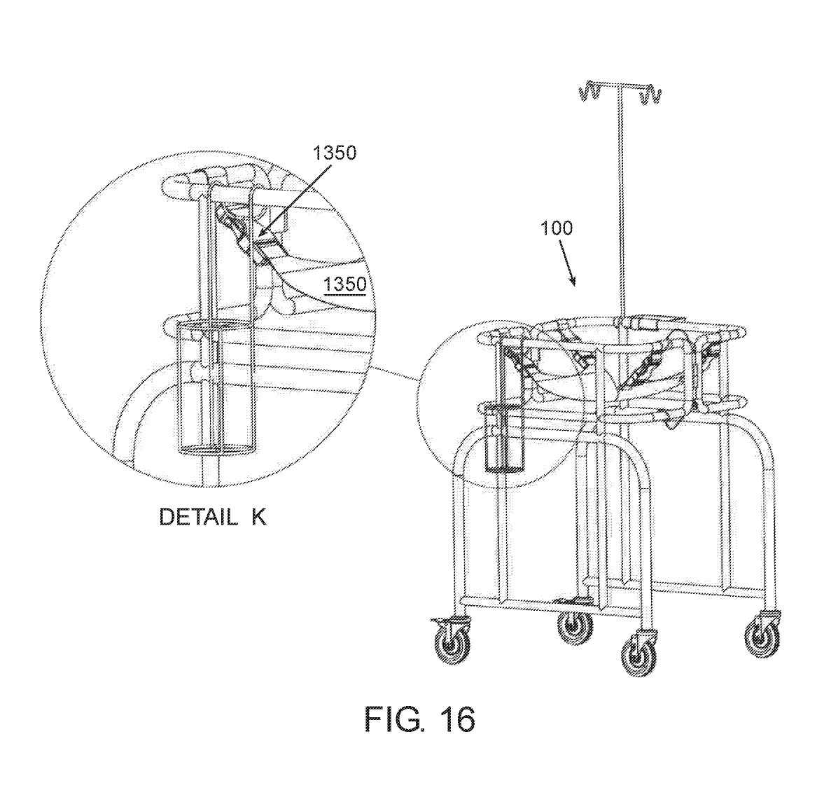

### DETAILED DESCRIPTION OF THE INVENTION

The present invention is directed to a medical transporter or medical transporter and methods thereof. The medical transporter provides complete safety and security for mobilization of a patient.

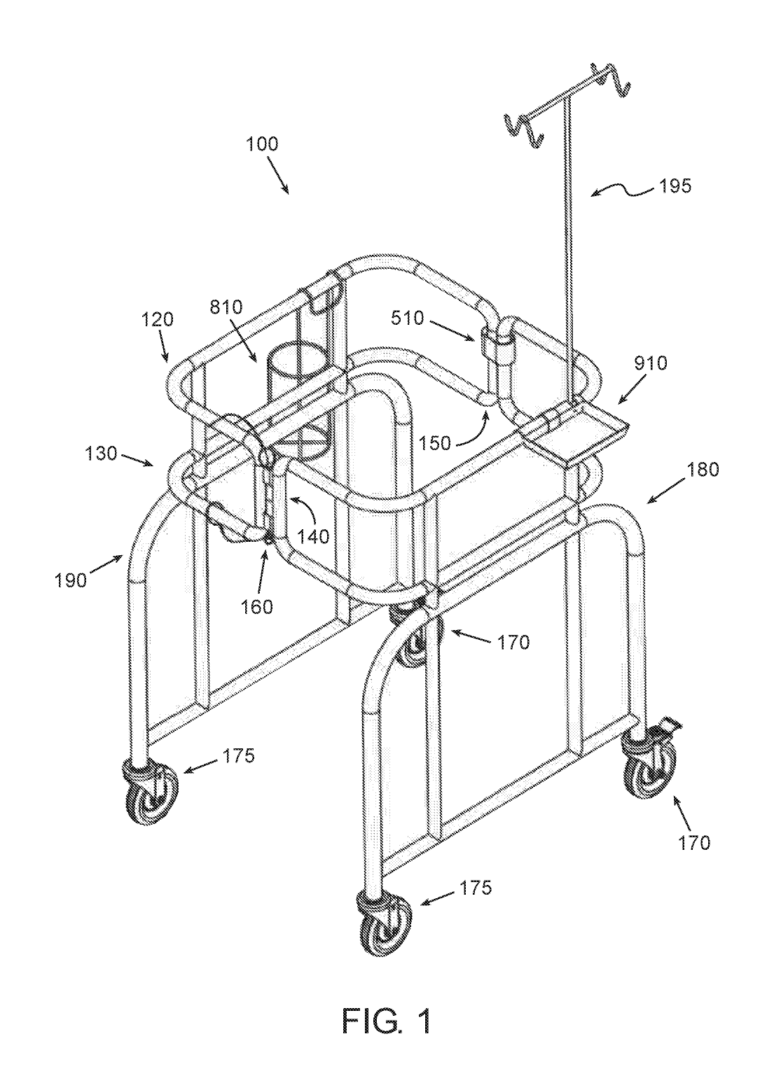

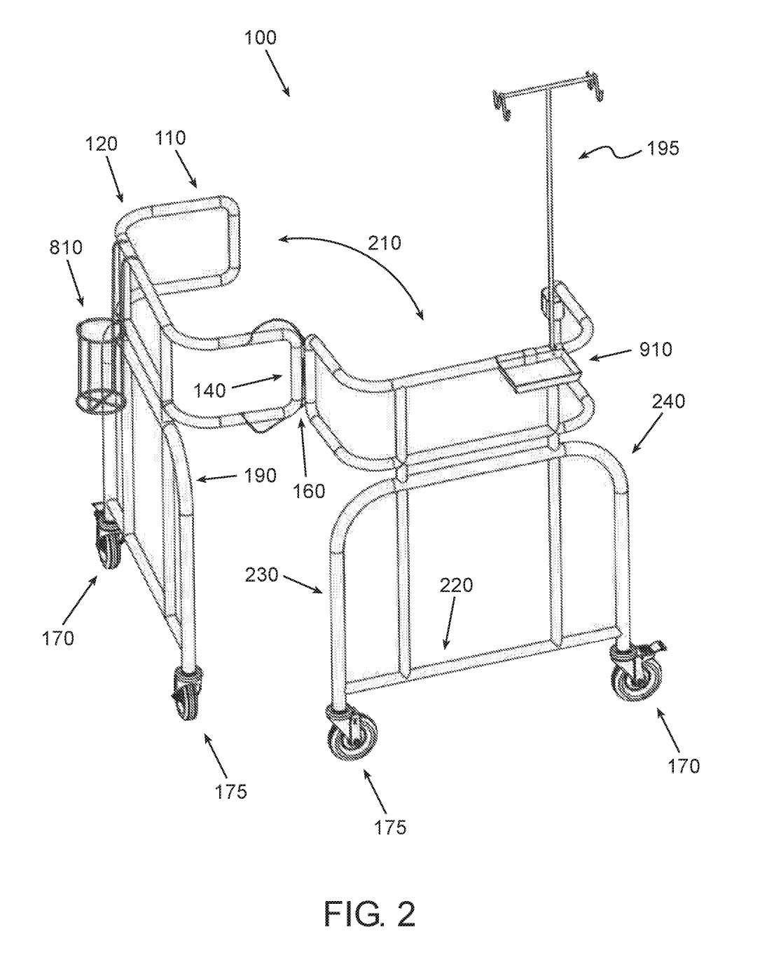

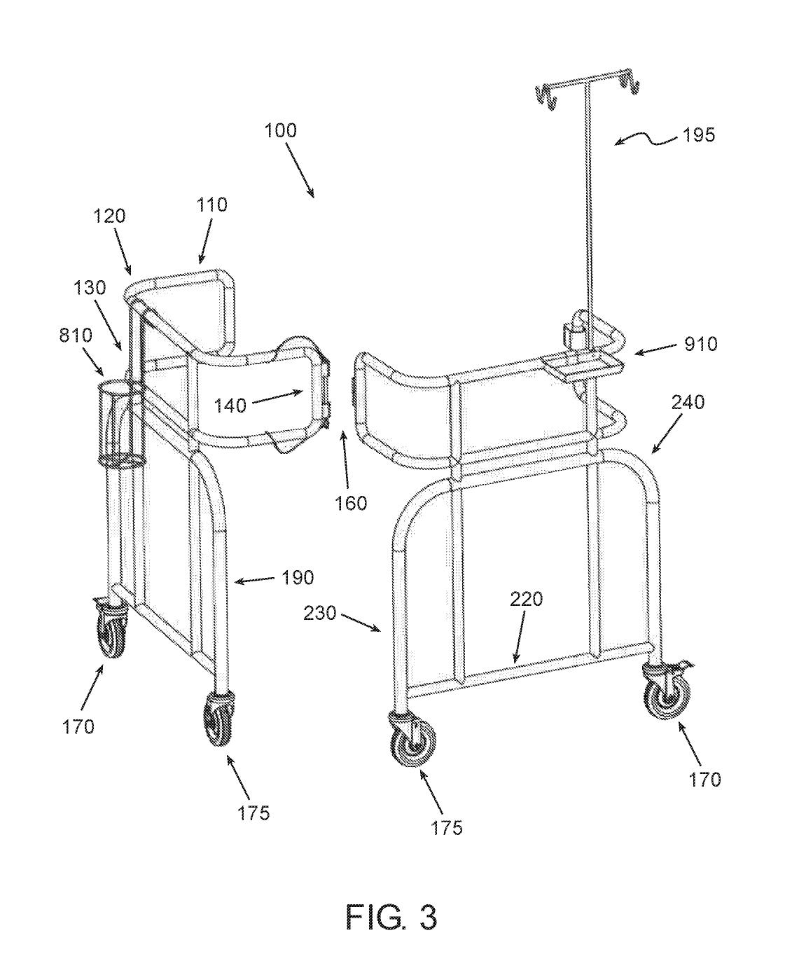

Referring to FIG. 1, a medical transporter (100) is provided including a support frame (110) said patient support frame comprising a first (120) and second (130) pairs of curved patient support rails, the second pair (130) of patient support rails being vertically spaced-apart from and located beneath said first pair of patient support rails (130) and connected to said first pair of patient support rails by respective pairs of front and back hollow vertical standards (140, 150). FIG. 1 also provides a hinge (160) pivotally joining together respective proximal ends of said first and second pairs of patient support rails (120, 130), and permitting movement of the patient support frame between a closed, patient encircling support position (as depicted in FIG. 1) and an open, patient ingress/egress position (as depicted in FIG. 2). The hinge (160) comprises a removable hinge pin (FIG. 3, 310) and end plate (320), said end plate (320) being connected to a one of said front vertical standards at a top end thereof and extending over a top end of the other of said front vertical standards, and said end plate (320) having a hole formed therein for permitting said hinge pin (310) to be inserted therethrough within the hollow body of the other of said front vertical standards, whereby said hinge pin (310) and said end plate (320) cooperate to permit pivoting movement of said patient support frame from the open and closed positions (as depicted by FIG. 2 arrow 210). The first and second leg frames (180, 190) may be attached to said patient support frame (110) and engaging the second pair of patient support rails (130) for vertically spacing the patient support frame above a supporting surface, and positioning the first pair of patient support rails (120, 130) generally in the region between the waist and upper torso of the patient. Wheels (170,175) carried by said first and second leg frames (180, 190) permits rolling movement of the medical transporter.

The patient support frame (100) further includes a second pair of patient support rails (130). The second pair of patient support rails are vertically spaced-apart from the first pair of patient support rails, and are connected to the first pair of patient support rails by respective pairs of front and back hollow standards (140, 150). The second pair of rails (130) are located below the first pair of rails (120) for respectively engaging the first and second leg frames.

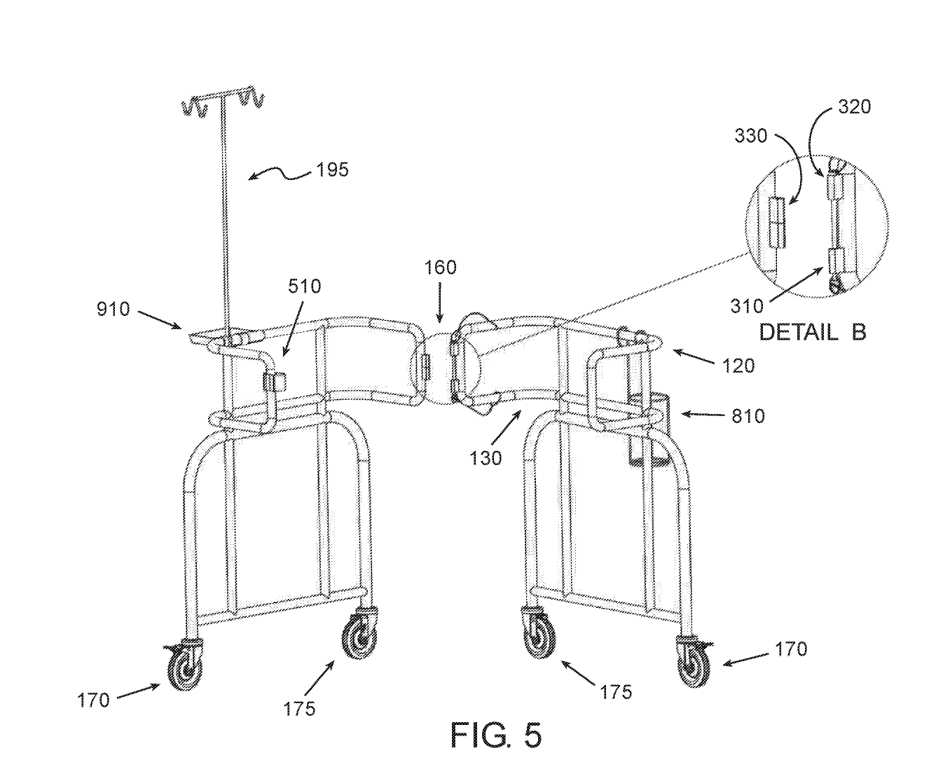

The medical transporter (100) may include a locking element (FIG. 5, 510) associated with the back pair of hollow vertical standards (150) for releasably locking said patient support frame in the closed position.

The locking element (510) may include first and second locking straps attached, respectively (not shown), to the first and second back vertical standards, the first strap may have a plurality of hook members located on an inner surface and a second strap having a plurality of complementary loop members located on an outer surface, whereby the first and second straps are locked together by mating the hook members of the first strap with the loop members of the second strap.

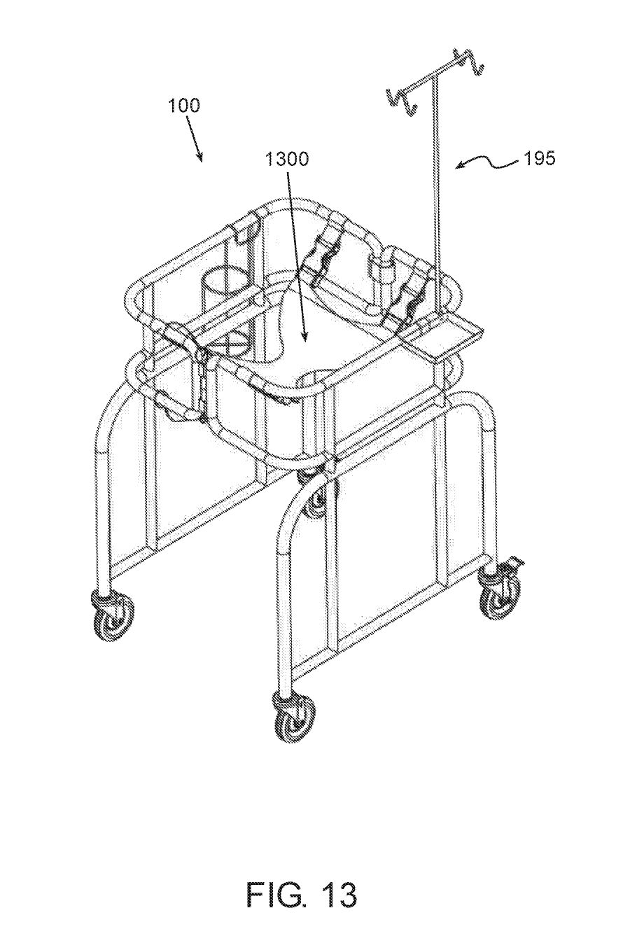

The medical transporter (100) may further include a fabric seat harness (1300, FIG. 13) suspended from the patient support frame for being positioned between the legs of the patient to support the patient in a seated position.

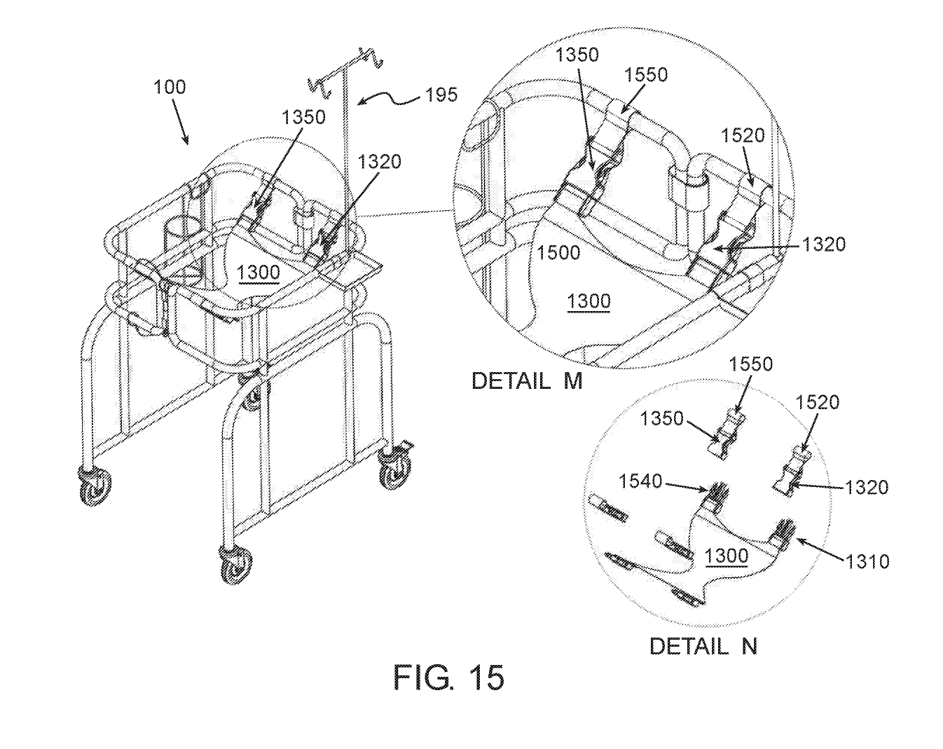

In certain embodiments, the fabric seat harness is connected to the patient support rails (120, 130) via a latch, backpack type latch (1540, FIG. 15), an airplane hanger latch and/or a male female connection, such that the connection is able to support the weight of a patient via the fabric seat.

In certain embodiments, the latch is connected to a hook or to a portion (1550, FIG. 15) that is looped around the frame.

The medical transporter (100) may include a horizontal bar (220) connected to a one of the rails of the second pair of patient support rails where each of the first and second leg frames (180, 190) includes a first and second hollow leg (230, 240) integrally formed with respective ends of the horizontal bar (220) and extending downwardly from said horizontal bar to the supporting surface, each of said hollow legs (230, 240) carrying respective wheels (170) for permitting rolling movement of the transporter.

The medical transporter (100) hollow legs (230, 240) may include a telescoping member (not shown) for permitting vertical adjustment of said patient support frame within the region of between the waist and upper torso of the patient.

The medical transporter (100) may also include a telescoping member (not shown) for adjusting the overall circumference of the patient support rails to accommodate users of varying size, to include the morbidly obese.

In certain embodiments, the frame is able to be reduced in size, such that it is adjustable within itself to shrink in size in diameter, so that patients of different sizes can use the medical transporter.

The medical transporter wheels (170) may include lockable castor wheels (175). The other transporter wheels (170) are shown not being lockable, but in certain embodiments, can be replaced with lockable castor wheels (175).

In certain embodiments, a foldable tray (910) and a removable basket (810) are provided. The foldable tray (910) is used to assist in transport of additional items and to provide a surface to place IV bags during replacement of the IV bags.

The removable basket (810) is configured to be used for storing and/or transporting IV bags, dosing pumps, oxygen tanks and/or concentrators, medical accessories and the like, for ease of transport with the patient.

In certain embodiments, the removable basket (810) is looped around the frame. In certain embodiments, the removable basket is made of a plastic or polypropylene material or a metal. In certain embodiments, the removable basket (810) is configured to be easily cleaned or sterilized.

In certain embodiments, one rail of the first pair of rails includes an upwardly extending bar (195) attached thereto for holding a medical IV bag in an elevated position generally above the chest of the patient.

In certain embodiments, a pulse medication dispenser (not shown) may be attached to the leg of a one of the leg frames.

An often occurrence and apprehension that occurs is that upon initial exertion, many patients have accidents from the bowels or urinary tract. The present invention is designed to offer dignity to the user with a diaper apparatus to alleviate this fear and embarrassment, while assisting in hygiene, clean up time and costs for the personnel.

Accordingly, this invention is not to be limited by the embodiments as described, since these are given by way of example only and not by way of limitation.

Having thus described several embodiments for practicing the inventive method, its advantages and objectives can be easily understood. Variations from the description above may and can be made by one skilled in the art without departing from the scope of the invention, which is to be determined from and by the following claims.

What is claimed is:

1. A medical transporter for transportation of a patient, comprising:
a frame, the frame configured to entirely enclose a patient during use of the medical transporter, said frame comprising:
a first frame member, the first frame member having an upper support member and a lower support member, the lower support member connected to a pair of wheels,
a second frame member, the second frame member having an upper support member, and a lower support member, the lower support member connected to a pair of wheels,
a hinge tethered on one end to the first frame member and on the other end to the second frame member, the hinge allowing the first frame member to pivot with respect to the second frame member,
a lock ring tethered on one end to the first frame member, the lock ring having a flexible receiving portion configured to interface with the second frame member, such that the lock ring holds the first frame member to the second frame member during use of the medical transporter; and
a removable stand, the removable stand being configured to support an IV drip bag, wherein the patient is able to transport the IV drip bag through transport of the medical transporter.

2. The medical transporter of claim 1, further comprising a removable seat, wherein the removable seat is secured to the first frame member and to the second frame member.

3. The medical transporter of claim 2, wherein the upper support member of the first frame member and the upper support member of the second frame member have connection members, the connection members configured to support the removable seat and the weight of the patent.

4. The medical transporter of claim 3, wherein the connection members are selected from the group consisting of hooks and rings.

5. The medical transporter of claim 2, wherein the removable seat is made a pliable polyester material or plastic material that is deformable and is configured to adapt to support the torso of the patient.

6. medical transporter of claim 1, wherein the frame is made of a metal, a metal alloy, steel or a material configured to support a patient up to 350 lbs.

7. The medical transporter of claim 2, further comprising elastic straps, the elastic straps configured to connect the removable seat to the first frame member and to the second frame member.

8. The medical transporter of claim 1, wherein the lower support member of the first frame member and the lower support member of the second frame member each comprise at least one reinforced structural component, the at least one reinforced structural component configured to provide additional support for the weight of the patient.

9. The medical transporter of claim 1, wherein at least one of the pair of wheels on the first frame member and the second frame member have locking members.

10. The transporter of claim 9, wherein the locking member is controlled via an actuator element located on the first frame member or the second frame member.

11. The medical transporter of claim 1, wherein the upper support member of the first frame member is curved and/or has a U-shape.

12. The medical transporter of claim 1, wherein the upper support member of the second frame member is curved and/or has a U-shape.

13. The medical transporter of claim 1, wherein the hinge further comprises a hinge pin.

14. The medical transporter of claim 1, wherein the removable stand is fitted to interface with the first or the second frame member.

15. The medical transporter of claim 1, wherein the hinge is welded into the first frame member and to the second frame member.

16. The medical transporter of claim 8, wherein the upper support member of the first frame member and the upper support member of the second frame member comprises a crossbar connected via the at least one reinforced structural component, wherein the at least one reinforced structural element is oriented vertically.

17. A medical transporter for transportation of a patient, comprising:
a frame, the frame configured to surround a patient during use of the medical transporter, said frame comprising:
a first frame member, the first frame member having an upper support member, a lower support member and a vertical standard, the lower support member connected to a pair of wheels and the upper support member connected to the lower support member via the vertical standard,
a second frame member, the second frame member having an upper support member, a lower support member and a vertical standard, the lower support member connected to a pair of wheels, and the upper support member connected to the lower support member via the vertical standard,
a hinge tethered on one end to the first frame member and on the other end to the second frame member, the hinge allowing the first frame member to pivot with respect to the second frame member, and
a locking element for releasably locking said patient support frame in a closed position during use of the medical transporter.

18. The medical transporter of claim 17, further comprising a first locking strap attached to the vertical standard of the first frame member, and a second locking straps attached to the vertical standard of the second frame member, the first strap locking strap having a plurality of hook members located on an inner surface and a second locking strap having a plurality of complementary loop members located on an outer surface, whereby the first locking strap and the second locking straps are locked together by mating the hook members of the first locking strap with the loop members of the second locking strap.

19. The medical transporter of claim 17, further comprising a removable stand, the removable stand being configured to support an IV drip bag, wherein the patient is able to transport the IV drip bag through transport of the medical transporter.

20. A method of transporting a patient in need thereof comprising:
inserting a patient into the medical transporter of claim 1, wherein the patient is inserted into the transporter when the transporter is in an open position;
closing the medical transporter around the patient; and
transporting the patient from a first location to a second location.
